(19) United States
(12) Patent Application Publication
(10) Pub. No.: US 2010/0069836 A1
(43) Pub. Date: Mar. 18, 2010

(54) RADIOFREQUENCY HOT BALLOON CATHETER

(75) Inventor: Shutaro Satake, Kanagawa (JP)

Correspondence Address: DARBY & DARBY P.C., P.O. BOX 770, Church Street Station, New York, NY, 10008-0770, US

(73) Assignee: JAPAN ELECTEL INC., Tokyo (JP)

(21) Appl. No.: 12/269,835

(22) Filed: Nov. 12, 2008

(30) Foreign Application Priority Data
Sep. 16, 2008 (JP) 2008-236984

(51) Int. Cl. A61B 18/08

(52) U.S. Cl. 604/96.01; 606/28; 604/114; 607/105

(57) ABSTRACT

A novel radiofrequency hot balloon catheter capable of exactly cauterizing a target site around the mitral annulus. The radiofrequency hot balloon catheter includes a catheter shaft comprising an outer cylinder shaft and an inner cylinder shaft which are mutually slidable, a balloon provided between vicinities of a distal end of the outer cylinder shaft and a distal end of the inner cylinder shaft, a liquid sending pathway formed between the outer cylinder shaft and the inner cylinder shaft to communicate with an inside of the balloon, a coil-shaped electrode provided inside the balloon and through which a radiofrequency current conducts for heating the inside of the balloon. On the catheter shaft in the vicinity of the balloon, the radiofrequency hot balloon catheter includes an intracardiac potential detection electrode 5a for detecting an intracardiac potential.

### BACKGROUND OF THE INVENTION

1. Field of the Invention

The present invention relates to a radiofrequency hot balloon catheter employed for cardiac affections, particularly for curing mitral regurgitation.

2. Description of the Related Art

Most of the mitral regurgitation is caused not by abnormality of a mitral valve itself but by a mitral annulus dilatation resulting from extension of tissues including an atrial wall around the mitral annulus. Therefore, the mitral regurgitation like this is cured by narrowing the mitral annulus through surgery. It has been, however, the problem that the surgery provided a large invasiveness.

For this reason, three approaches to the therapy for the mitral regurgitation have been developed in which an implantable device is left inside a heart by using a cardiac catheter to narrow the mitral annulus. One approach involves utilizing a ring-shaped device with a stent which constrains the coronary sinus to reduce the cross-sectional area of the mitral annulus. Another approach involves utilizing a device for clipping anterior and posterior leaflets of the mitral annulus. And yet another approach involves utilizing a stapler-type device for reefing atrial muscles just beneath the mitral annulus (e.g., refer to nonpatent documents, “Prospects for Percutaneous Valve Therapies, Feldman T, Leon M B. Circulation. 2007:116; 2866-2877, and “Mitral Apparatus: Functional Anatomy of Mitral Regurgitation. Perloff J K, Roberts W C. Circulation 1972; 46; 227-239). When either approach is practiced, however, due to leaving an artificial device inside a cardiac chamber, disengagement of the device is likely to cause serious complications and besides in order to prevent a complication of thromboembolism, an antithrombotic or an anticoagulant must be used over long periods.

Therefore, for the purpose of solving the above problem, approaches to the therapy for the mitral regurgitation, which dispenses with accompanying surgery and the use of the implantable device, is being sought. As one of these approaches, an approach can be thought of in which tissues around the mitral annulus is cauterized using a radiofrequency hot balloon catheter (e.g., refer to International publication No. WO 2007-052341 pamphlet, Japanese unexamined patent publication Nos. 2008-167958, 2005-177293 and 2004-223080).

From the anatomical standpoint, the mitral valve is composed of an anterior leaflet and a posterior leaflet and then a basal portion of the anterior leaflet continues into an aortic wall, while the posterior leaflet continues into a left atrial free wall. Most of the mitral regurgitations are attributable mainly to the disorder that the left atrial free wall is extended and thereby the mitral annulus is displaced into a side of a left atrium to be enlarged. Consequently, it is considered that if the left atrial free wall which is enlarging the mitral annulus and the tissues around the mitral annulus are selectively cauterized to be subjected to scar contraction and thereby the mitral annulus is narrowed to return to its original position, the mitral regurgitation can be cured.

When employing the conventional radiofrequency hot balloon catheter, however, a balloon positional relation to the mitral annulus was hard to exactly grasp in the case of cauterizing selectively the tissues around the mitral annulus. Accordingly, it has been the problem that a target site around the mitral annulus was difficult to exactly cauterize.

### SUMMARY OF THE INVENTION

Therefore, in view of the problem described above, it is an object of the present invention to provide a novel radiofrequency hot balloon catheter capable of exactly cauterizing a target site around a mitral annulus.

According to a first aspect of the present invention, there is provided a radiofrequency hot balloon catheter which is employed to reduce the cross-sectional area of the mitral annulus to cure mitral regurgitation by cauterizing a left atrial wall adjacent to the mitral annulus enlarged and tissues around the left atrial wall from sides of the left atrial wall and coronary sinus to be subjected to scar contraction.

According to another aspect of the present invention, a radiofrequency heating catheter is equipped with a catheter shaft comprising an outer cylindrical shaft and an inner cylindrical shaft which are mutually slidable, a balloon provided between vicinities of a distal end of the outer cylindrical shaft and distal end of the inner cylindrical shaft, a liquid sending pathway formed between the outer cylindrical shaft and the inner cylindrical shaft to communicates with an inside of the balloon, a radiofrequency current conducting electrode which is provided inside the balloon and through which a radiofrequency current conducts for heating the inside of the balloon, an vibration generator which generates oscillating waves, a temperature sensor which detects central temperature inside the balloon, a radiofrequency generator which feeds radiofrequency electric power to the radiofrequency current conduction electrode, and further an intracardiac potential detection electrode which is provided on the catheter shaft in the vicinity of the balloon to detect an intracardiac potential, and an intracardiac potential recorder which records an intracardiac potential detected by the intracardiac potential detection electrode.

According to another aspect of the present invention, the vibration generator is equipped with an oscillating wave conduction changeover switch which switches between a conduction state and interruption state of oscillating waves to the liquid sending pathway.

According to another aspect of the present invention, the radiofrequency generator is equipped with a feedback circuit which maintains the central temperature inside the balloon at a preset value and besides decreases the preset value of the central temperature inside the balloon when a contact surface between the balloon and biomedical tissues decreases and thereby an output of the radiofrequency electric power considerably rises.

According to another aspect of the present invention, the radiofrequency generator is equipped with a relay circuit for stopping the radiofrequency electric power from being fed when the central temperature inside the balloon does not reach 60° even if an output of the radiofrequency electric power is maximized.

According to another aspect of the present invention, the intracardiac potential recorder is equipped with a safety device which emits a warning sound or stops the radiofrequency electric power from being fed to the radiofrequency conducting electrode when a ventricular potential is higher than an atrial potential.

According to another aspect of the present invention, the intracardiac potential detection electrode is formed from a radiopaque material.

According to another aspect of the present invention, a film of an approximately spherical or spindle-shape central portion in the balloon is 20 to 50 μm in thickness and a film of a basal portion in the balloon is 50 μm or more in thickness.

According to another aspect of the present invention, the intracardiac potential detection electrode is made of iron.

According to another aspect of the present invention, the radiofrequency heating catheter is equipped with a guide sheath which includes the intracardiac potential detection electrode at its distal end and is flexible and further the catheter shaft and the balloon can be shoved into an inside of the sheath.

According to another aspect of the present invention, therapy for mitral regurgitation according to the present invention employs the radiofrequency heating catheter.

According to the radiofrequency hot balloon catheter of the present invention, the mitral annulus is narrowed to permit the mitral regurgitation to be cured.

Further, the intracardiac potential detecting electrode for detecting the intracardiac potential is provided on the catheter shaft in the vicinity of the balloon. Hence, it becomes possible by detecting the intracardiac potential to exactly grasp a positional relation to the mitral annulus and as a result the biomedical tissues at the target site can be exactly cauterized.

Furthermore, the vibration generator is equipped with the oscillating wave conduction changeover switch which switches between a conduction state and interruption state of the oscillating waves to the liquid sending pathway. When the oscillating waves have been interrupted, the inside of the balloon is not agitated. Hence, a heating operation at an upper portion of the inside of the balloon is accelerated by thermal convection, thus enabling only the biomedical tissues in contact with an upper half portion of the balloon to be selectively cauterized.

Moreover, the radiofrequency generator is equipped with the feedback circuit which maintains the central temperature inside the balloon at the preset value and decreases the preset value of the central temperature inside the balloon when the contact surface between the balloon and the biomedical tissues has decreased and thereby an output of the radiofrequency electric power considerably rises. When decreasing the contact surface of the balloon with the biomedical tissues, a contact surface with a bloodstream increases to cool the balloon by the bloodstream and when maintaining the central temperature inside the balloon, so the output of the radiofrequency electric power considerably rises. Therefore, a temperature difference decreases between the central temperature inside the balloon and the temperature at the contact surface of the balloon. At this time, by decreasing the preset value of the central temperature inside the balloon, the contact surface of the balloon with the biomedical tissues can be prevented from excessively rising.

Besides, the radiofrequency generator is equipped with a relay circuit for stopping the radiofrequency electric power from being fed when the central temperature inside the balloon does not reach 60° even if the output of the radiofrequency electric power is maximized. When the balloon is not in contact with the biomedical tissues, even if the radiofrequency electric power is maximized, the central temperature inside the balloon does not reach 60°. At this time, by stopping the radiofrequency electric power from being fed, a redundant heating operation can be restrained.

Further, the intracardiac potential recorder is equipped with the safety device which emits the warning sound or stops the radiofrequency electric power from being fed to the radiofrequency conduction electrode when a ventricular potential is higher than an atrial potential. The balloon could have cauterized the mitral valve at a ventricular side when the ventricular potential is higher than the atrial potential. At this time by emitting the warning sound or stopping the radiofrequency electric power from being fed, the mitral valve at the ventricular side can be prevented form being cauterized.

Furthermore, the intracardiac potential detection electrode is formed from the radiopaque material. Hence, a balloon position can be fine adjusted by a radio-opacity.

Moreover, the film of the approximately spherical or spindle-shaped central portion in the balloon is 20 to 50 μm in thickness and the film of the basal portion therein is 50 μm or more in thickness. Hence, heat inside the balloon can be efficiently conducted to the biological tissues.

Further, the intracardiac potential detection electrode is made of iron. Hence, by utilizing, together with the balloon, a catheter equipped with a magnet at its distal end, the balloon can be firmly attached to tissues by utilizing the magnet.

Furthermore, the radiofrequency hot balloon catheter is equipped with a guide sheath which includes the intracardiac potential detection electrode at its distal end and besides is flexible and further the catheter shaft and the balloon can be shoved into an inside of the sheath. Hence, by detecting the intracardiac potential, it becomes possible to exactly grasp the positional relation of the distal end of the guide sheath to the mitral annulus and by inflecting the guide sheath, the balloon can be exactly attached firmly to the biometrical tissues at the target site.

According to the therapy for mitral regurgitation, the mitral regurgitation can be certainly cured by utilizing the radiofrequency hot balloon catheter.

### BRIEF DESCRIPTION OF THE DRAWINGS

These objects and other objects and advantages of the present invention will become more apparent upon reading of the following detailed description and the accompanying drawings in which:

FIG. 1 is an explanatory view illustrating a structure in the vicinity of a radiofrequency hot balloon catheter according to the present embodiment.

FIG. 2 is an explanatory view illustrating an overall structure and its usage state of the radiofrequency hot balloon catheter according to the present embodiment.

FIG. 3 is an explanatory view illustrating a cross-section in the vicinity of a balloon of the radiofrequency hot balloon catheter according to the present embodiment.

FIG. 4 is a graph illustrating temporal changes in output of a radiofrequency generator, in central temperature of the balloon and in contact temperature of the balloon in the radiofrequency hot balloon catheter according to the present embodiment.

FIG. 5 is a graph illustrating temporal changes in output of a radiofrequency generator, in temperatures at an upper portion of the balloon and at a lower portion of the balloon, and contact temperature of the balloon in the radiofrequency hot balloon catheter according to the present embodiment.

### DETAILED DESCRIPTION OF THE PREFERRED EMBODIMENT

The present invention provides a radiofrequency hot balloon catheter which can be employed for curing mitral regurgitation through narrowing a mitral annulus by selectively cauterizing, from a side of a left atrial endocardium and a side of a coronary sinus endocardium, a left atrial free wall which is enlarging the mitral annulus and tissues around the mitral annulus to thereby be subjected to scar contraction.

Hereinafter, one embodiment of the radiofrequency hot balloon catheter according to the present invention is described in detail with reference to accompanying drawings.

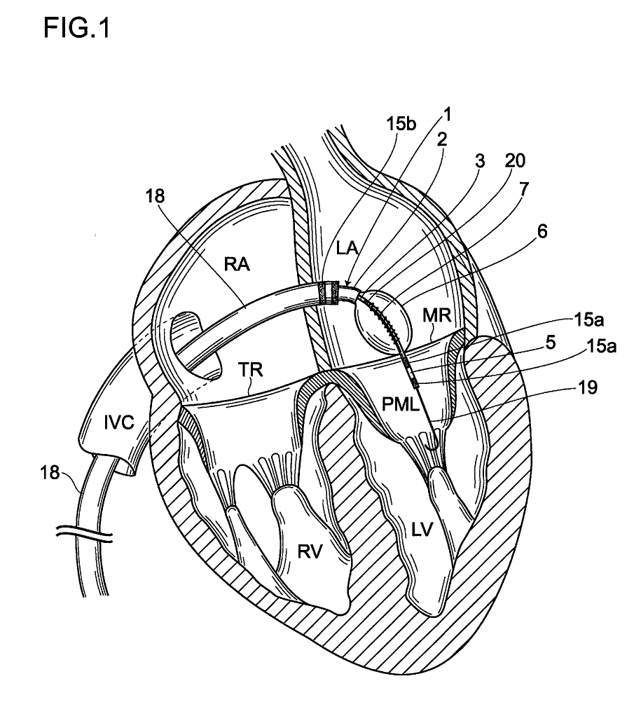

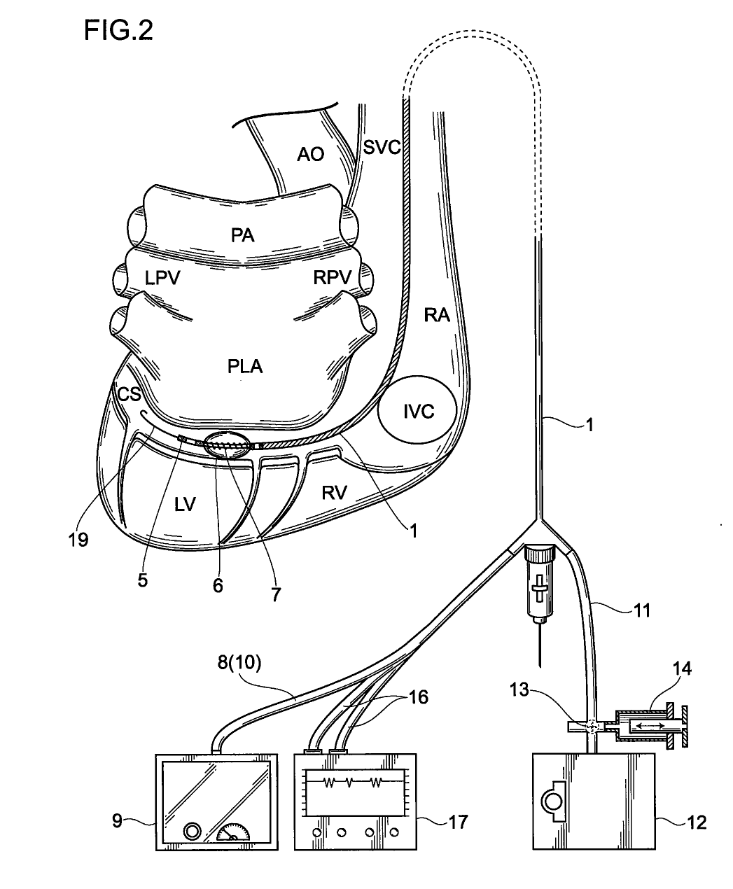

With reference to FIG. 1 and FIG. 2, described is a structure of the radiofrequency hot balloon catheter according to the present embodiment.

Numeral symbol 1 denotes a catheter shaft, which comprises an outer cylindrical shaft 2 and an inner cylindrical shaft 3 which are mutually slidable. A balloon 6 is provided between vicinities of a distal end 4 of the outer cylindrical shaft 2 and distal end 5 of the inner cylindrical shaft 3. Then, the catheter shaft 1 and the balloon 6 can be shoved into an inside of a guide sheath 18 described later.

The balloon 6 is formed in an approximately spherical or an approximately spindle-like shape. A film of a central portion of the balloon 6 is made 20 to 50 μm in thickness and a film of a basal portion thereof is made 50 μm or more in thickness. By thus thinning the film thickness of the central portion contacting biological tissues, heat inside the balloon 6 is allowed to effectively transfer to the biological tissues, while by thickening the film thickness not contacting the biological tissues, the heat inside the balloon 6 is allowed to be hard to dissipate to a bloodstream.

A coil-shaped electrode 7, acting as a radiofrequency current conducting electrode, through which a radiofrequency current conducts for heating an inside of the balloon 6 is wound around the inner cylindrical shaft 3 to be provided inside the balloon 6. Then, a radiofrequency generator 9, which feeds the radiofrequency current to the coil-shaped electrode 7, is connected with the coil-shaped electrode 7 via a radiofrequency current carrying wire 8. Besides, a thermo couple 20, acting as a temperature sensor for detecting central temperature inside the balloon 6, is provided inside the balloon 6. Further, a thermometer (not shown) provided in the radiofrequency generator 9 is connected with the thermo couple 20 via a conductive wire 10. In addition, the radiofrequency current carrying wire 8 and the conductive wire 10 reach the balloon 6 through an inside of the catheter shaft 1. The radiofrequency electric power fed to the coil-shaped electrode 7 and the temperature detected by the thermo couple are schemed so as to be displayed on the radiofrequency generator 9. Furthermore, the radiofrequency generator 9 is equipped with a control means (not shown) which automatically regulates the radiofrequency electric power so as to maintain the central temperature inside the balloon 6 at a preset value based on the temperature detected by the thermo couple while measuring impedance of a circuit containing the coil-shaped electrode 7. The control means 9 is equipped with a feedback circuit which lowers the preset value of the central temperature inside the balloon 6 when a contact area between the balloon 6 and the biomedical tissues had decreased and thereby an output of the radiofrequency electric power has considerably risen and with a relay circuit which stops the radiofrequency electric power from being fed when the central temperature inside the balloon 6 does not reach 60° even if the output of the radiofrequency electric power is maximized.

A liquid sending pathway (not shown) which communicates with the inside of the balloon 6 is formed between the outer cylindrical shaft 2 and the inner cylindrical shaft 3. Accordingly, liquid is sent to the balloon 6 through the liquid sending pathway to enlarge the balloon 6. An vibration generator 12 which generates an oscillating wave is connected with the liquid sending pathway via an oscillating wave transmission duct 11. Further, an oscillating wave transmission changeover switch 13 which switches between a transmission state and interruption state of the oscillating wave to the liquid sending pathway is provided at a connection of the vibration generator 12 with the oscillating wave transmission duct 11. So, when the oscillating wave transmission changeover switch 13 is switched to a side of the transmission state to transmit the oscillating wave, vortex flows are generated by the oscillating wave generated by the vibration generator 12 to agitate the liquid inside the balloon 6, thus maintaining uniformly the temperature inside the balloon 6. On the contrary, when the oscillating wave transmission changeover switch 13 is switched to a side of the interruption state to interrupt the oscillating wave, the inside of the balloon 6 is not allowed to be agitated. Furthermore, a syringe 14 for introducing the liquid to the liquid sending pathway is provided at the connection of the vibration generator 12 with the oscillating wave transmission duct 11.

An intracardiac potential detection electrode 15a, which detects an intracardiac potential and is made of iron, is provided at a distal end of the catheter shaft 1 in the vicinity of the balloon 6, that is, a distal end 5 of the inner cylindrical shaft 3. Iron is a radiopaque material and so, by obtaining a radio-opacity, a position of the balloon 6 can be fine adjusted. As the intracardiac potential detection electrode 15a is made of iron, by utilizing, together with the balloon 6, a catheter equipped with a magnet at its distal end to take advantage of magnetic force, the balloon 6 can be attached firmly to the biomedical tissues. Further, an intracardiac potential recorder 17 which records an intracardiac potential detected by the intracardiac potential detection electrode 15a is connected with the intracardiac potential detection electrode 15a via the conductive wire 16. When a ventricular potential is higher than an atrial potential, the balloon 6 could have cauterized the mitral valve at a ventricular side. In order to prevent the mitral valve at the ventricular side from being cauterized, when the ventricular potential is higher than the atrial potential, the intracardiac potential recorder 17 is equipped with a safety device which emits a warning sound or stops the radiofrequency electric power from being fed to the radiofrequency conduction electrode 7.

A guide sheath 18 is provided on a periphery of the outer cylindrical shaft 2 and a distal end of the guide sheath 18 is allowed to be flexible. Then, by inflecting the distal end of the guide sheath 18, the balloon 6 is allowed to be attached firmly to biomedical tissues at a target site. Besides, a guide wire 19 is inserted into the inner cylindrical shaft 3. A distal end of the guide wire 19 is formed in a U-shape.

The distal end of the guide sheath 18 is provided with an intracardiac potential detection electrode 15b which detects the intracardiac potential and is made of iron. By utilizing the intracardiac potential detection electrode 15b and the intracardiac potential detection electrode 15a at the same time, the position of the balloon 6 can be more exactly fine adjusted. Then, the intracardiac potential recorder 17 is connected with the intracardiac potential detection electrode 15b as is the case with the intracardiac potential detection electrode 15a.

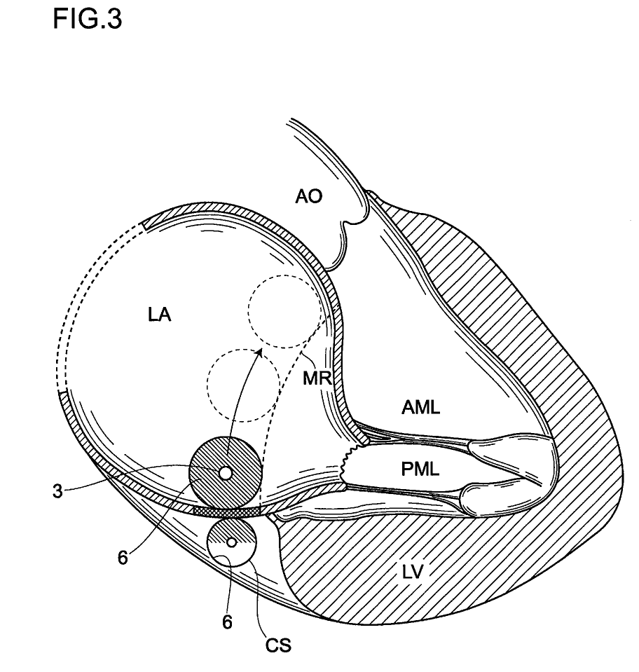

Next, with reference to FIG. 1 to FIG. 3, behavior of the radiofrequency hot balloon catheter is described with therapy for mitral regurgitation taken as an example. By using the Brockenbrough method, the sheath 18 is first inserted into femoral vein to burst through an atrial septum from a right atrium (RA) and reach the left atrium, thus inserting the guide sheath 18 into the left atrium (LA). A U-shaped distal end of the guide wire 19 is made to stay inside a left ventricle (LA) or a left pulmonary vein (LPV) under radioscopy and subsequently the balloon 6 is guided by the guide wire 19 to be inserted into the left atrium (LA). Then, the balloon 6 is enlarged to be allowed to become in contact with a posterior wall of the left atrium (PLA) with indications given by radio-opacities of the intracardiac potential detection electrodes 15a, 15b and by intracardiac potentials detected by the intracardiac potential detection electrodes 15a, 15b. Further, clockwise rotary torque is applied to the guide sheath 18 to attach a lateral side of the balloon 6 firmly to the posterior wall of the left atrium (PLA).

Here, if the distal end of the balloon 6 is at the mitral valve (MR), the atrial potential and the ventricular potential which have been detected by the intracardiac potential detection electrodes 15a are equal substantially in wave height, while if the distal end of the balloon 6 is at a side of the left atrium (LA), the atrial potential is higher than the ventricular potential and further if being at a side of the left ventricle (LV), the ventricular potential is higher than the atrial potential. In a similar fashion, if the distal end of the guide sheath 18 is at the mitral valve (MR), the atrial potential and the ventricular potential which have been detected by the intracardiac potential detection electrode 15b are substantially equal in wave height, while if the distal end of the guide sheath 18 is at a side of the left atrium (LA), the atrial potential is higher than the ventricular potential and further if being at a side of the left ventricle (LV), the ventricular potential is higher than the atrial potential. Consequently, by laying the balloon 6 at a position where the atrial potential is higher than the ventricular potential, the balloon 6 can be surely attached firmly to the posterior wall of the left atrium (PLA). As a result, in the following cauterizing operation, only the left atrium (LA) can be selectively cauterized, while the mitral valve at the side of the left ventricle (LV) can be surely prevented from being erroneously cauterized. In addition, it is difficult that the position of the balloon 6 is maintained at an exact position at the side of the atrium only by utilizing the radioscopy.

Then, a radiofrequency current with 50 to 150 W is started to be applied to the coil-shaped electrode 7 to raise the central temperature of the balloon 6 to 60 to 75° C. and that temperature is kept unchanged for 3 to 5 minutes. At this time, the vibration generator 12 is activated to agitate the liquid inside the balloon 6, equalizing the temperature inside the balloon 6. Then, shifting the position of the balloon 6 little by little, the whole of the posterior wall of the left atrial connecting to a posterior mitral leaflet (PML) is cauterized. At this time, when another balloon 6 is inserted into a coronary sinus (CS) lying along the posterior wall of the left atrial (PLA) to be enlarged therein and thereby the bloodstream is blocked off, a cauterization effect is enhanced in the posterior wall of the left atrial (PLA).

Subsequently, the balloon 6 lied inside the coronary sinus (CS) for blocking off the bloodstream is displaced to a position where the atrial potential detected by the intracardiac potential detection electrode 15a is higher than the ventricular potential and then the radiofrequency current is applied to the coil-shaped electrode 7. At this time, when switching the oscillating wave transmission changeover switch 13 to the side of the interruption state to block off the oscillating wave, only an upper portion of the balloon 6 is heated by thermal convection and then in a patient lying face up, only an upper wall of a coronary sinus (CS) and a side of the posterior wall of the left atrial (PLA) in contact with the coronary sinus (CS) are selectively cauterized. The sites cauterized change into fiber tissues after one to two months and the mitral valve (MR) is narrowed by its scar contraction, thus improving the mitral regurgitation.

In addition, the balloon 6 produces an effect chiefly by thermal conduction and hence a cauterizing depth increases in proportion to the temperature of the biomedical tissue in contact with the balloon 6 and electric conduction duration. Accordingly, thickness of a wall of the left atrium (LA) is preliminarily measured by an intercardiac ultrasonic device. Then, the central temperature inside the balloon 6 and the electric conduction duration are set depending on the thickness measured and thereby only a target site can be selectively cauterized.

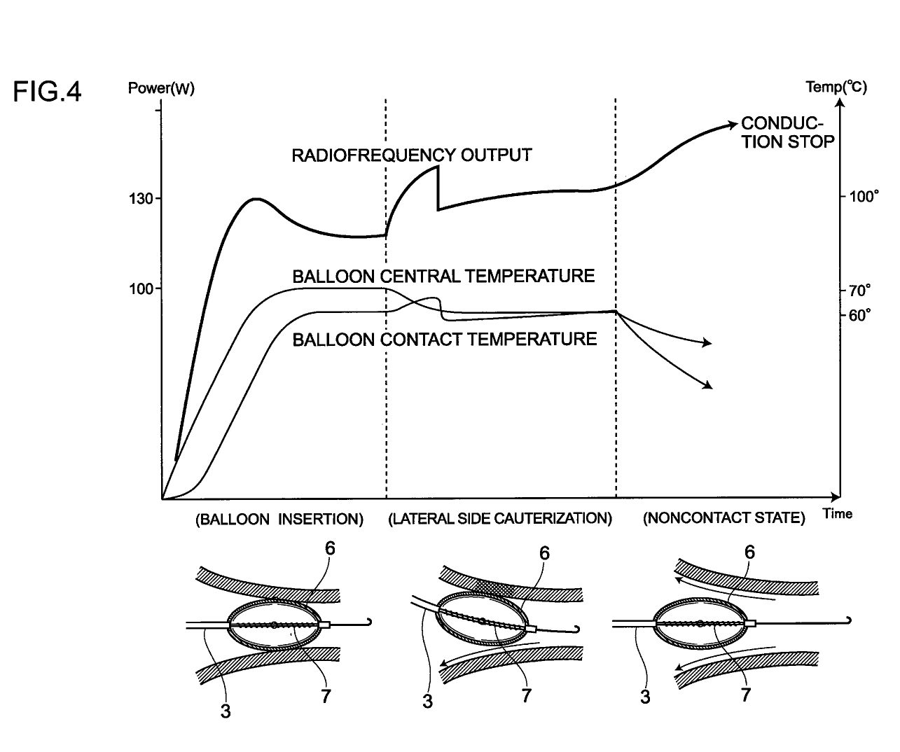

In FIG. 4, the changes over time in the radiofrequency output power of the radiofrequency generator 9, in the central temperature of the balloon 6 and in the temperature at the site of the biomedical tissues in contact with the balloon 6 is graphed out.

When switching a cauterizing operation by using the whole of a lateral circumferential surface of the balloon 6 to that by using only one lateral side of the balloon 6, the contact surface between the balloon 6 and the biomedical tissues decreases and as a result the balloon 6 is considerably cooled by the bloodstream. Then, the control means widely increases the radiofrequency output in order to hold the central temperature of the balloon 6 constant to decrease a difference between the central temperature of the balloon 6 and the temperature of the contact surface of the balloon 6 increases. Then, the preset value of the central temperature inside the balloon 6 is decreased by the feedback circuit. This operation of the feedback circuit restrains the temperature of the contact surface of the balloon 6 from excessively rising.

Further, when having become in no contact with the biomedical tissues, the balloon 6 is considerably cooled by the bloodstream and thereby even if the radiofrequency output is maximized, the central temperature inside the balloon 6 does not reach 60°. At this time, the radiofrequency electric power is stopped by the relay circuit from being fed. So, the operation of the relay circuit prevents redundant heating.

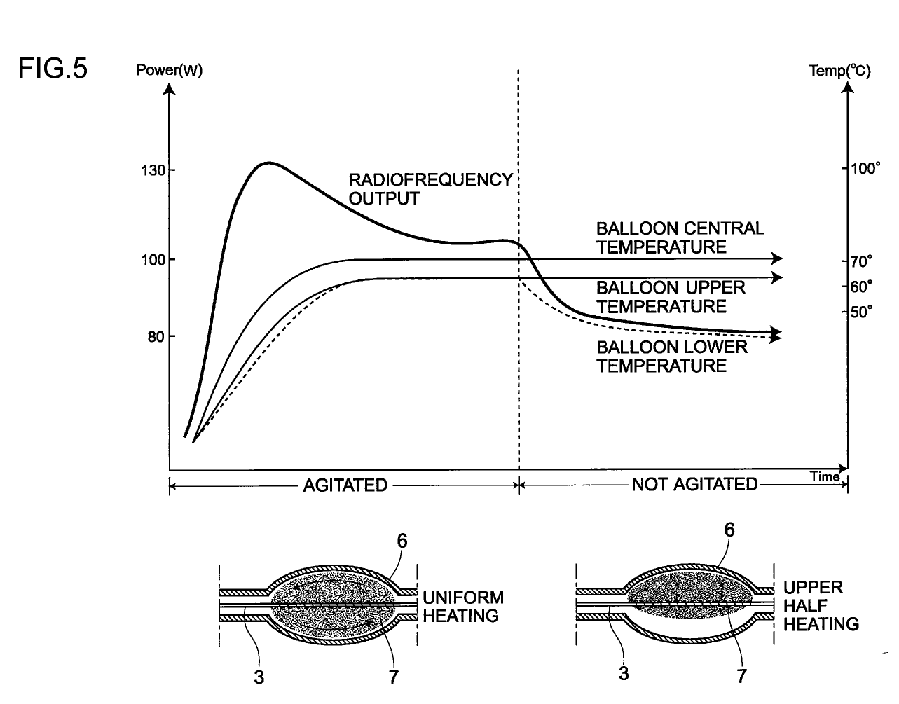

In FIG. 5, the changes over time in the radiofrequency output power of the radiofrequency generator 9, in the central temperature of the balloon 6, in the temperature of the upper portion of the balloon 6, and in the temperature of the lower portion of the balloon 6 is graphed out.

When switching the oscillating wave transmission changeover switch 13 to the side of the transmission state to transmit the oscillating wave, the oscillating wave generated by the vibration generator 12 generates vortex flows inside the balloon 6 to agitate the liquid inside the balloon 6. At this time, the temperatures at the upper and lower portions of the balloon 6 become equal to each other, thus holding the temperature inside the balloon 6 uniform.

When switching the oscillating wave transmission changeover switch 13 to the side of the interruption state to block off the oscillating wave, the inside of the balloon 6 is not agitated. At this time, although the temperature at the upper portion of the balloon 6 is kept constant by thermal convection, the temperature at the lower portion of the balloon 6 decreases.

As described above, the radiofrequency hot balloon catheter according to the present embodiment is equipped with the catheter shaft 1 comprising the outer cylindrical shaft 2 and the inner cylindrical shaft 3 which are mutually slidable, the balloon 6 provided between the vicinities of the distal end 4 of the outer cylinder shaft 2 and distal end 5 of the inner cylinder shaft 3, the liquid sending pathway formed between the outer cylindrical shaft 2 and the inner cylindrical shaft 3 to communicates with the inside of the balloon 6, the coil-shaped electrode 7, acting as the radiofrequency current conducting electrode, which is provided inside the balloon 6 and through which the radiofrequency current conducts for heating the inside of the balloon 6. Besides, in the radiofrequency hot balloon catheter, the intracardiac potential detection electrode 15a is provided on the catheter shaft 1 in the vicinity of the balloon 6 to detect the intracardiac potential. Accordingly, the positional relation of the balloon 6 to the mitral annulus can be exactly grasped by detecting the intracardiac potential, so that the biomedical tissues at the target site can be exactly cauterized.

Further, the radiofrequency hot balloon catheter according to the present embodiment is equipped with the vibration generator 12 which generates the oscillating wave. The vibration generator 12 is equipped with the oscillating wave transmission changeover switch 13 which switches between the transmission and interruption states of the oscillating wave to the liquid sending pathway. When the oscillating wave has been interrupted, the inside of the balloon 6 is not agitated. As a result, convection heat accelerates heating at the upper portion of the balloon 6. Accordingly, only the biomedical tissue in contact with the upper half portion of the balloon 6 can be selectively cauterized.

Furthermore, the radiofrequency hot balloon catheter according to the present embodiment is equipped with the thermo couple which detects the central temperature inside the balloon 6 and with the radiofrequency generator 9 which feeds radiofrequency electric power to the coil-shaped electrode acting as the radiofrequency current conducting electrode. The radiofrequency generator 9 is equipped with the feedback circuit which maintains the central temperature inside the balloon 6 at the preset value and besides decreases the preset value of the central temperature inside the balloon 6 when the output of the radiofrequency electric power has considerably increased. When the contact surface of the balloon 6 with the biomedical tissues is decreased, the contact surface of the balloon 6 with the bloodstream is increased and then if aiming at maintaining the temperature inside the balloon 6, the output of the radiofrequency electric power is considerably increased. At this time, by decreasing the preset value of the central temperature inside the balloon 6, the temperature at the contact surface of the balloon 6 with the biomedical tissues can be prevented from excessively rising.

Moreover, the radiofrequency hot balloon catheter according to the present embodiment is equipped with the thermo couple which detects the central temperature inside the balloon 6 and the radiofrequency generator 9 which feeds the radiofrequency electric power to the coil-shaped electrode 7 acting as the radiofrequency current conducting electrode. The radiofrequency generator 9 is equipped with the relay circuit which stops the radiofrequency electric power from being fed when the central temperature inside the balloon 6 does not reach 60° even if the output of the radiofrequency electric power is maximized. When the balloon 6 is not in contact with the biomedical tissues, the central temperature inside the balloon 6 does not reach 60° even if the output of the radiofrequency electric power is maximized. At this time, by stopping the radiofrequency electric power from being fed, redundant heating can be restrained.

Besides, the radiofrequency hot balloon catheter according to the present embodiment is equipped with the intracardiac potential recorder 17 which records the intracardiac potential detected by the intracardiac potential detection electrode 15a. Further, the intracardiac potential recorder 17 is equipped with the safety device which emits the warning sound or stops the radiofrequency electric power from being fed to the coil-shaped electrode 7 when the ventricular potential is higher than the atrial potential. When the ventricular potential is higher than the atrial potential, the balloon 9 could have cauterized the mitral valve at the side of the ventricle. At this time, by emitting the warning sound or stopping the radiofrequency electric power from being fed to the coil-shaped electrode 7 acting as the acting as the radiofrequency current conducting electrode, the mitral valve at the side of the ventricle can be prevented from being cauterized.

Further, the intracardiac potential detection electrode 15a is formed from the radiopaque material. Accordingly, the radio-opacity enables the position of the balloon 6 to be fine adjusted.

Furthermore, the film of the approximately spherical or spindle-shaped central portion in the balloon 6 and the film of the basal portion therein are 20 to 50 μm and 50 μm or more in thickness, respectively. Accordingly, the heat inside the balloon 6 can be efficiently transmitted to the biomedical tissues.

Moreover, the intracardiac potential detection electrode 15a is made of iron. Accordingly, by utilizing, together with the balloon 6, the catheter equipped with the magnet at its distal end, the balloon 6 can be attached firmly to the biomedical tissues by using magnetic force.

Besides, the radiofrequency hot balloon catheter according to the present embodiment is equipped with the intracardiac potential detection electrode 15b and besides the guide sheath 18 which is flexible. The catheter shaft 1 and the balloon 6 can be shoved into the inside of the guide sheath 18. Accordingly, by detecting the intracardiac potential, the positional relation of the distal end of the guide sheath 18 to the mitral annulus can be exactly grasped and further by inflecting the guide sheath 18, the balloon 6 can be exactly attached firmly to the biomedical tissues of the target site.

In addition, the present invention is not limited to the embodiment described above and various modifications are possible within the gist of the scope of the invention. The radiofrequency heating catheter according to the present invention can be applied not only to the therapy for the mitral regurgitation but to therapies for tricuspid valve insufficiency, aortic valve regurgitation and pulmonary insufficiency. Further, it is possible to cauterize the whole of the posterior wall of the left atrium including coronary sinus (CS) and thereby cure atrial fibrillation caused by the posterior wall of the left atrium. Furthermore, the radiofrequency hot balloon catheter can be applied not only to the therapy for cardiac affection but to therapy for gastroesophageal reflux disease and thermal therapies for esophageal cancer, stomach cancer, large intestine cancer, pulmonary cancer, or the like.

What is claimed is:

1. A radiofrequency hot balloon catheter employed to cure mitral regurgitation with reduction of the caliber of the mitral annulus, wherein said radiofrequency hot balloon catheter cauterizes the left atrial wall and the coronary sinus with scar contraction of the left atrium and the surrounding tissue adjacent to the enlarged mitral annulus.

2. The radiofrequency hot balloon catheter according to claim 1, comprising:
a catheter shaft comprising an outer cylinder shaft and an inner cylinder shaft which are mutually slidable;
a balloon provided between vicinities of an distal end of said outer cylinder shaft and distal end of said inner cylinder shaft;
a liquid sending pathway formed between said outer cylinder shaft and said inner cylinder shaft to communicate with an inside of said balloon;
a radiofrequency current conducting electrode which is provided inside said balloon and through which a radiofrequency current conducts for heating the inside of said balloon;
a vibration generator which generates oscillating waves;
a temperature sensor which detects central temperature inside said balloon;
a radiofrequency generator which feeds radiofrequency electric power to said radiofrequency current conducting electrode;
an intracardiac potential detection electrode which is provided on said catheter shaft in the vicinity of said balloon to detect an intracardiac potential; and
an intracardiac potential recorder which records an intracardiac potential detected by said intracardiac potential detection electrode.

3. The radiofrequency hot balloon catheter according to claim 2, wherein said vibration generator is equipped with an oscillating wave transmission changeover switch which switches between a conduction state and interruption state of oscillating waves to said liquid sending pathway.

4. The radiofrequency hot balloon catheter according to claim 2, wherein said radiofrequency generator maintains central temperature inside said balloon at a preset value and is equipped with a feedback circuit for decreasing a preset value of central temperature inside said balloon when a contact area between said balloon and the vital tissues decreases and thereby an output of radiofrequency electric power considerably increases.

5. The radiofrequency hot balloon catheter according to claim 2, wherein said radiofrequency generator is equipped with a relay circuit for stopping radiofrequency electric power from being fed when central temperature inside said balloon does not reach 60° even if an output of radiofrequency electric power is maximized.

6. The radiofrequency hot balloon catheter according to claim 2, wherein said intracardiac potential recorder is equipped with a safety device which emits warning sounds or stops radiofrequency electric power from being fed to said radiofrequency conducting electrode when a ventricular potential is higher than an atrial potential.

7. The radiofrequency hot balloon catheter according to claim 2, wherein said intracardiac potential detection electrode is formed from a radiopaque material.

8. The radiofrequency hot balloon catheter according to claim 2, wherein a film of an approximately spherical or spindle-shaped central portion in said balloon is 20 to 50 μm in thickness and a film of a basal portion in said balloon is 50 μm or more in thickness.

9. The radiofrequency hot balloon catheter according to claim 2, wherein said intracardiac potential detection electrode is made of iron.

10. The radiofrequency hot balloon catheter according to claim 2, wherein said radiofrequency hot balloon catheter is equipped with a guide sheath which includes an intracardiac potential detection electrode at its distal end and besides has a flexible structure and further said catheter shaft and said balloon can be shoved into an inside of said guide sheath.

11. Therapy for mitral regurgitation, wherein said therapy employs the radiofrequency heating catheter according to claim 1.

12. The therapy for mitral regurgitation, wherein said therapy employs the radiofrequency heating catheter according to claim 2.

13. The therapy for mitral regurgitation, wherein said therapy which employs the radiofrequency heating catheter according to claim 3.

14. The therapy for mitral regurgitation, wherein said therapy which employs the radiofrequency heating catheter according to claim 4.

15. The therapy for mitral regurgitation, wherein said therapy which employs the radiofrequency heating catheter according to claim 5.

16. The therapy for mitral regurgitation, wherein said therapy employs the radiofrequency heating catheter according to claim 6.

17. The therapy for mitral regurgitation, wherein said therapy employs the radiofrequency heating catheter according to claim 7.

18. The therapy for mitral regurgitation, wherein said therapy employs the radiofrequency heating catheter according to claim 8.

19. The therapy for mitral regurgitation, wherein said therapy employs the radiofrequency heating catheter according to claim 9.

20. The therapy for mitral regurgitation, wherein said therapy employs the radiofrequency heating catheter according to claim 10.
